(19) United States
(12) Patent Application Publication
(10) Pub. No.: US 2023/0390397 A1
(43) Pub. Date: Dec. 7, 2023

(54) SYSTEM, METHOD AND PROCESS FOR SELF-STERILIZATION OF IODINE-CONTAINING DEVICES

(71) Applicant: HDR LLC, Park City, UT (US)

(72) Inventors: Minh HOANG, Las Vegas, NV (US); Bryan Garrett DAVIS, Sandy, UT (US)

(21) Appl. No.: 18/201,717

(22) Filed: May 24, 2023

Related U.S. Application Data
(60) Provisional application No. 63/345,383, filed on May 24, 2022.

(51) Int. Cl. A61K 41/10; A61L 2/00; A61K 33/18

(52) U.S. Cl. CPC A61K 41/10; A61L 2/0023; A61L 2/0088; A61K 33/18; A61M 35/003

(57) ABSTRACT

A method and system for sterilizing an iodine antiseptic solution is disclosed. The iodine antiseptic solution can comprise iodine crystals and/or povidone-iodine (PVP-I). The method includes providing the iodine antiseptic solution to a closed container. And during a sterilization cycle, the method further includes heating the iodine antiseptic solution in the closed container to a sterilization temperature sufficient to generate free elemental iodine in the closed container. The method further includes applying a positive pressure in the closed container to pressurize the free elemental iodine to produce sterilized iodine within the closed container.

### CROSS-REFERENCE TO RELATED APPLICATIONS

This application claims the benefit of U.S. Provisional Application No. 63/345,383, filed May 24, 2022. This application is incorporated herein by reference in its entirety.

### BACKGROUND

Regulations regarding the sterilization requirements of antiseptic solutions vary considerably around the world. In some jurisdictions, such as most European Union (EU) countries, some degree of sterilization is required. However, in the United States, there are currently no regulations for the sterilization of antiseptic solutions, and therefore, antiseptic solutions currently sold in the United States generally do not undergo a sterilization process. For example, currently, patient preoperative skin preparations are not required to be sterile.

In December 2012, the Food and Drug Administration (FDA) sought to obtain input on how to address microbial contamination of patient preoperative skin preparation products. In July 2021, the FDA advised drug manufacturers of non-sterile, water-based drug products that the Burkholderia cepacia complex (BCC or “B. cepacia”) continued to pose a serious risk of contamination of such products. At that time, there were many incidents of antiseptic products, such as chlorhexidine gluconate, povidone iodine and chloroxylenol, among others, being contaminated with bacteria. Bacteria can contaminate these products at the time of manufacture, during storage, or during product use. Contaminated patient preoperative skin preparations have been associated with clinical infections and adverse outcomes. Thus, there is a need to have antiseptic solutions be sterilized.

Several known antiseptic solutions currently be used in a hospital setting include: 2% to 4% w/v chlorhexidine gluconate in water or in 70% v/v alcohol in water; povidone iodine solution with the povidone iodine in the range 0.5% to 10% in water or with 70% alcohol in water; chloroxylenol (PCMX) in the range of 3-4% with surfactant in water. Conventional methods of sterilization of antiseptic drug products include heat sterilization (i.e., by autoclave), ethylene oxide sterilization, and gamma radiation sterilization etc.

Regardless of which method is used to sterilize the antiseptic products, the antimicrobial molecules will degrade by the method, which creates undesired impurities as well as lowers the overall concentration of the active drug moiety. The high concentration of impurities may be toxic for human use and also may be carcinogenic. The lower the active drug concentration may even cause the product to be recalled due to the product not meeting the product label's claim. Regulations in at least the United States and EU countries limit the quantity of impurities that may be present in an antiseptic solution, especially after the sterilization.

Electron beam sterilization (E-beam) is a form of radiation sterilization using beta particles to neutralize or kill microbes. Gamma radiation is another form of radiation sterilization using the self-disintegration of Cobalt-60 (60Co) or Cesium-137 to neutralize or kill microbes. Sterilization by gamma radiation and E-beam have been known to be effective sterilizing processes but are notoriously unsuitable for most antiseptic products. For instance, gamma radiation breaks down polymers, chemical components, and active ingredients (such as chlorhexidine, povidone iodine, and chloroxylenol) of some products and creates many impurities. In the U.S., any antiseptic products sterilized by gamma radiation are required to submit an application to the FDA to be approved before marketing these products. This regulation process is known as a New Drug Application (NDA) application. The NDA process requires 5-10 years to be approved by the FDA and the company must spend huge sums of investment to conduct all the testing requirements per NDA. Therefore, the industry stays away from gamma radiation sterilization for antiseptics products.

Ethylene oxide (ETO) sterilization is used, almost exclusively, for medical device sterilization, which devices are packaged with breathable packaging. ETO gas cannot permeate through a thick plastic packaging or rigid closed container. However, ETO sterilization is not suitable for antiseptic solutions which are packaged in a plastic chamber, glass ampoule, a syringe, a form-fill-seal unit closed container, or the like.

Another sterilizer, known as an “autoclave,” uses saturated steam at 121-132° C. in a carbon steel container for sterilization. A typical standard for steam sterilization is achieved after 15 to 30 minutes under a pressure of 106 kPa (1 atm) once all surfaces have reached a temperature of 121° C. The autoclaving process takes advantage of the phenomenon that the boiling point of water (or steam) increases when it is under high pressure, as can be seen in FIG. 1. However, autoclave systems and processes have several distinct disadvantages, such as only being applicable for small batches of solutions to be sterilized and not suitable for mass production, as well as moisture retention, some effects of which are described below.

The carbon steel can get damaged due to exposure to moisture, chemical components, certain polymers, and other active ingredients, and can degrade under high temperature and under high pressure, etc. Using an autoclave, only stainless-steel instruments and plastics that can bear high heat be sterilized. Several plastic components and packaging of device containers cannot handle such heat, such as prefilled drug syringes containing a drug that is sensitive to high temperature. Several iodine products currently on the market are not stable under high temperature due to poor formulation. Accordingly, high temperature sterilization with an autoclave is not suitable due to expected degradation.

Free elemental iodine (I₂ ) is a well-known antimicrobial agent having activity of a broad-spectrum antiseptic with bactericidal, fungicidal, sporicidal and virucidal properties. A few parts per million (ppm) in solution is sufficient to kill bacteria and viruses. Iodine-based products currently in use rely on free elemental iodine as the main antimicrobial agent. These products may also be formulated with cationic, anionic or non-ionic or surfactants and skin emollients for skin or topical application purposes.

Elemental iodine (I₂ ) was first isolated as an antimicrobial. At room temperature it is a dark purple, lustrous, crystalline solid. Upon heating it melts to form liquid at 113.5° C. and boils to a pinkish purple vapor at 184.4° C., but can sublime to vapor directly from the solid depending on conditions. Iodine dissolves readily in ethanol or ether to produce brown solutions, or in chloroform or benzene as violet-colored solutions. It is sparingly soluble in water (0.29 g/l at 20° C., 0.33 g/l, 1.2 mM, at 25° C., 0.78 g/l at 50° C. and 1.25 g/l at 80° C.) yielding a yellowish-brown solution. Solubility of elemental iodine increases in the presence of iodide ions, such as potassium iodide, where iodine reacts to form tri-iodide ions.

One of the most widely used iodine-containing solutions, or iodophors, in hospitals is povidone-iodine (PVP-I), which is often used in an iodophor solution as a bactericide. It has also been used as a patient skin preparation prior to a surgical procedure or for surgical hand scrubbing. Aqueous-based iodophors, such as povidone-iodine, contain iodine complexed with a solubilizing agent, allowing for the release of free iodine when in a solution. Iodine acts in an antiseptic manner by destroying microbial proteins and DNA. Iodophor-containing products have widespread use because of their broad-spectrum antimicrobial properties, efficacy, and safety on nearly all skin surfaces regardless of the patient's age.

As to the mechanism of action and antimicrobial spectrum of iodine, as a small molecule, iodine rapidly penetrates into microorganisms and oxidizes key proteins, nucleotides, and fatty acids, eventually leading to cell death. PVP-I has a broad antimicrobial spectrum with activity against gram-positive and gram-negative bacteria, including antibiotic-resistant and antiseptic-resistant strains, fungi, and protozoa. It is also active against a wide range of enveloped and nonenveloped viruses, as well as some bacterial spores with increased exposure time. In addition, PVP-I has shown to have activity against mature bacterial and fungal biofilms in vitro and ex-vivo.

However, there is an unmet need in the art for a method of heat sterilizing iodine solutions that has a shorter sterilization time at low temperature, more efficient processing time, and provides a sterile solution while maintaining sufficient purity of the iodine solution to comply with regulatory requirements and do not create more impurities.

The activity of antimicrobial agents is always influenced by pH, temperature, concentration, contact time, presence of organic matter, electrolytes, microbial strains and neutralizers used. For all the iodine solutions after the manufacturing or on the market, these products are already fixed by the formulation, fixed concentration of the active ingredients but these products are considered as nonsterile products due to the contamination of bacteria in the final solution during mixing, manufacturing, or bacteria in the raw material components.

Many different types of plastic have differing melting points. A wide variety of common plastics begin to melt at 100 degrees Celsius (212° F.). For example, polyethylene (PE) is a soft polymer found in two main types: low density PE (LDPE) and high-density PE (HDPE). At higher temperatures, PE loses its rigidity and begins to melt. LDPE will already begin melting at 105° C. and HDPE will begin melting at 125° C. Polypropylene (PP) is a slightly harder and stiffer plastic than HDPE plastic, it therefore has a higher melting point of 165° C. Polystyrene (PS) is a hard polymer used in the manufacture of well-known polystyrene foam. PS does not require a high melting point, which is why it melts at around

Plastic materials subjected to prolonged exposure to high temperatures will lose strength and toughness, becoming more prone to cracking, chipping, and breaking, at a rate in proportion to the temperature and time of exposure. Materials exposed to higher heat for longer duration will wear substantially faster than those exposed to more moderate temperatures and exposure times.

Therefore, there is further a need to sterilize the final device or container containing the iodine solutions before using these devices in a medical procedure to prevent HAIs when using these products.

### SUMMARY

This document describes systems, methods and processes for self-sterilization of iodine-containing solutions and devices that contain such solutions. Systems and methods described herein focus on the influence of pressure and temperature to increase the antimicrobial activity of the antimicrobial agent-free iodine in the iodine solutions to make devices become sterile products through a process call a self-sterilization process. The systems and methods described herein cover all iodine solutions.

In accordance with implementations described herein, a sterilization process utilizes the heating of a closed container system at low temperature (60-100 C) to produce free elemental iodine under high pressure (i.e., greater than atmosphere) and utilizing the high degree of diffusion of elemental iodine through water, air and lipids, and its reactivity as an oxidizing agent against bacteria and viruses. Elemental iodine is used as an antimicrobial to kill any contamination by bacteria in the iodine solution, especially when heating the container that contains iodine solution in a range of 60-100° C. At higher temperature, a chemical reaction will produce more free elemental iodine and the free elemental iodine created from heating the iodine solution kills all the bacteria in the solution and in the container surface. Accordingly, this is a process of self-sterilization. This self-sterilization process using heat in the range of 60 to 100° C. within a short period of time, i.e., 5-30 minutes does not create any new impurities for the iodine solution.

In one aspect, and consistent with the disclosure herein, a method for sterilizing an iodine antiseptic solution is described. The iodine antiseptic solution includes iodine crystals and/or povidone-iodine (PVP-I). The method includes the step of providing the iodine antiseptic solution to a closed container. The method further includes the steps, during a sterilization cycle, of heating the iodine antiseptic solution in the closed container to a sterilization temperature sufficient to generate free elemental iodine in the closed container, and applying a positive pressure in the closed container to pressurize the free elemental iodine to produce sterilized iodine within the closed container.

In another aspect consistent with the disclosure herein, a system for disinfecting epithelium of a living organism is described. The system includes a device having a surface configured for being placed in proximity to the epithelium of the living organism. The system further includes a container in contact with the device, the container containing sterilized iodine and being pressurized to a positive pressure. The container is configured to release the sterilized iodine under the positive pressure to disinfect the surface of the device prior to or while the surface is placed in proximity to the epithelium of the living organism to disinfect the epithelium in proximity thereof.

In yet another aspect consistent with the disclosure herein, a sterilization system for sterilizing an iodine antiseptic solution is described. The system includes a plurality of containers, each of the plurality of closed containers containing a portion of the iodine antiseptic solution. The system further includes a chamber configured to host the plurality of containers, the chamber further being configured to execute a sterilization cycle to sterilize the portion of the iodine antiseptic solution in each of the plurality of closed containers. As described herein, the sterilization cycle comprises heating the iodine antiseptic solution in each of the plurality of containers to a sterilization temperature sufficient to generate free elemental iodine in each container, and applying a positive pressure in each of the plurality of containers to pressurize the free elemental iodine to produce sterilized iodine within each container.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 is a phase diagram for water to illustrate the effects of heat and temperature on an aqueous solution;

FIG. 2 is a flowchart of a method of self-sterilization of a solution; and

FIGS. 3A-3C show various containers that hold an iodine-containing solution for being subject to the method of sterilization described herein.

Like reference symbols in the various drawings indicate like elements.

### DETAILED DESCRIPTION

This document describes systems, methods and processes for self-sterilization of iodine-containing solutions, and devices or systems that contain or utilize such solutions.

Aqueous solutions of iodine are not stable and, depending on conditions, many different species of an iodine solution may be present. Of these, molecular iodine (I₂) may have the highest antimicrobial potential. Stability is influenced by pH, and activity, such as antimicrobial activity, diminishes with increased alkalinity and storage time. As to how iodine behaves chemically, its reactions in water are described below. The seven principal iodine species found in aqueous solution are I₂, HOI, OI⁻, H₂OI⁺, I₃⁻, I⁻ and IO₃⁻, of which only hydrated iodine (I₂ ), Hypoiodous acid (HOI) and iodine cation (H₂OI⁻⁾, possess bactericidal activity. At physiologically compatible pH levels and low concentrations, the only species of importance for an antiseptic solution are I⁻, I₂ and I₃⁻.

In accordance with implementations described herein, a sterilization system and process utilize the heating of a closed container system at low temperature (60-100 C), and under higher pressure of the atmosphere, to produce free elemental iodine. The utilizing of a high degree of diffusion of elemental iodine through water, air and lipids, and its reactivity as an oxidizing agent against bacteria and virus, elemental iodine is used as antimicrobial to kill the contamination bacteria in the iodine solution, especially when heating the container contains iodine solution in the range of 60-100° C. At higher temperatures the chemical reaction will produce more free elemental iodine, and the free elemental iodine creating from the heating the iodine solution will kill all the bacteria in the solution and in container surface. Accordingly, this is referred to as a “self-sterilization” process. This self-sterilization process in the range of 60 to 100° C. with short period of time 5-30 minutes do not create any new impurity for the iodine solution.

Temperatures above 65° C. (149° F.) can cause near complete inactivation of viruses and bacteria with exposures greater than 3 minutes. For temperatures between 55° C. and 60° C. (131-140° F.) heating should last 5 minutes or more. However, for temperatures in the range 50-55° C. (122-131° F.) 20 minutes or longer of exposure are needed. At these levels, the viral concentration can be expected to be lowered by log 5-7, near or below the detectable limit. Because of the seriousness of the current virus and bacterial infection, a safety factor for the product sterility can be obtained by increasing the above-listed temperatures by 10° C. (about 18° F.) so the sterilization temperature is 75° C. The sterilization temperature is in the range of 60-100° C. prefer range 70-80° C. and the sterilization time is 3 minutes to 24 hours, prefer range 10-30 minutes.

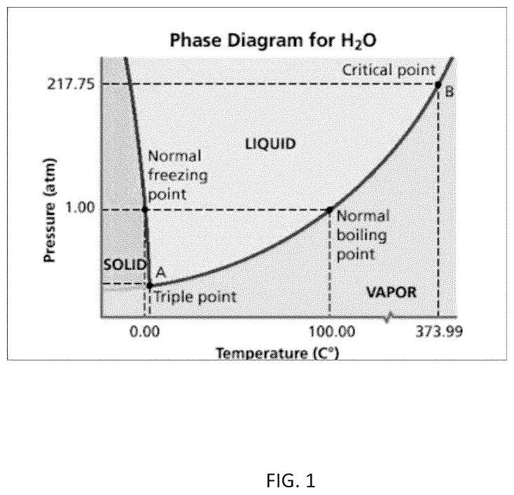

FIG. 1 is a phase diagram for water, to illustrate the effects of heat and temperature on an aqueous solution, such as an aqueous solution that can contain an iodine solution. Each sterilization process includes one or more phases, for example: 1) Phase I, a conditioning phase. In this phase the sterilizer temperature and pressure are slowly brought up to a desired temperature and pressure; 2) Phase II, a sterilization phase. This phase is also known as the holding time phase where the sterilizer is held at a specific temperature and pressure; and 3) Phase III, bringing the sterilizer in a safe state to open. In this phase, the temperature and pressure of the sterilizer is brought to room temperature and pressure.

In accordance with implementations described herein, a number of carriers for iodine can be used. For example, in specific implementations, four types of carriers for iodine can be used: poloxamer iodophors, cationic surfactant iodophors, nonionic surfactant iodophors and polyvinyl-pyrrolidone iodophors (otherwise known as polyvinyl-pyrrolidine-iodine, povidone iodine or PVP-I). In most of these agents, iodine is carried in aggregates (or micelles) of a surfactant, and which act as reservoirs of iodine. On dilution, these micelles slowly disperse to release free elemental iodine in aqueous solution, so that the concentration of the active agent gradually increases without reaching the undesirable concentrations associated with former products. This free iodine is known as available iodine and the activity of the iodophors is related to the amount of iodine released.

Iodophors are solutions that contain povidone-iodine (PVP-I), a stable chemical complex of polyvinylpyrrolidone (povidone, PVP) and elemental iodine. With these solutions, and under certain environmental constraints or factors, a small amount of iodine is slowly released in solution. They are typically used at concentrations ranging from 6 to 75 ppm depend on the formula. Iodophors penetrate the cell walls and membranes of microorganisms and interfere with DNA synthesis. Iodophors also bind to proteins, causing their inactivation. Traditional aqueous-based iodophors, such as povidone-iodine, are one of the few products that can be safely used on animal mucous membrane surfaces. The equilibrium concentrations of I₂ and I₃⁻ in aqueous povidone-iodine solutions (0.001-20.0%, pH 4, 25‡ C) have been evaluated from the redox potential and the iodide concentration as measured by the iodide electrode (HOI, OI⁻H₂O⁺I and IO₃⁻ can be neglected under the chosen conditions).

The values obtained for [I⁻], [I₂], [I₃⁻] and Cₒₓ (i.e., iodometrically titrable iodine solutions) indicate that the amount of iodine which is complex bound to the povidone matrix consists of HI3 and I₂ groups. At concentrations>1%, it represents nearly the whole oxidation capacity, while can be neglected below 0.01%. In some implementations, the concentration of the free, molecular iodine (I₂ ) only comes to 4.5×10-m/l (1.1 ppm) in the 20% solution and increases to a maximum of˜10-4 m/l (25.4 ppm) in the 0.1% solution.

The iodide ion (I⁻) present in the PVP-I solution is converted to iodine (I₂ ), and this free iodine molecule acts as an antimicrobial agent to kill any bacteria it contacts. Further, this iodine molecule can impregnate into any material that it is exposed to or to which it contacts, to make these materials themselves become antimicrobial. Accordingly, these materials, such as integrated with a medical device, can have one or more antimicrobial features, such as killing any bacteria that comes into contact with the surface of the medical device. Additionally, this antimicrobial material prevents any bacteria from building on a surface, such as on a biofilm on the surface of the medical device or material forming the same. All formulations of PVP-I are not the same, and the rate of release of free iodine is dependent on the components of a formulation. For instance, some components of a formulation will support the solubility of free iodine in the solution, while some components may slow down the rate of release of free iodine into the solution.

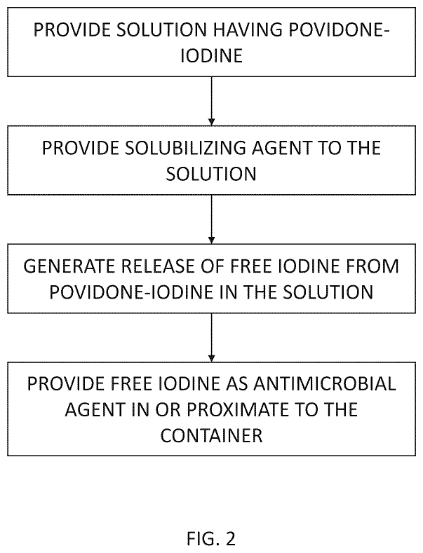

FIG. 2 is a flowchart of a method for self-sterilization of an iodine-containing container, such as a syringe, pouch, jar, or other container. At 202, a solution having povidone-iodine is provided to a container. Preferably, the solution takes up the entirety of the volume of the container, so as to not leave any space or air in the container. At 204, a solubilizing agent is provided to the solution, as described herein. At 206, a release of free iodine from the povidone-iodine is generated in the solution, by chemical reactions as described herein. At 208, the free iodine is provided as an antimicrobial agent in or proximate to the container, such as entirely within the container, and/or outside the container by osmosis or other chemical process (especially if the container includes an iodine-permeable membrane).

The method of self-sterilization described herein is sufficient to cover any iodine-containing solutions. The activity of antimicrobial agents is always influenced by, without limitation, pH, temperature, concentration, contact time, presence of organic matter, electrolytes, microbial strains and/or neutralizers used in any particular solution. Current iodine-containing solutions on the market, i.e., after manufacturing, while fixed in their formulations and active ingredients, are considered non-sterile due to potential contamination of bacteria in the final stages of mixing the material components and in manufacturing a device that contains the solution, either from contamination after the manufacturing process or during the mixing or manufacturing process due to contamination of one or more raw material components.

Accordingly, implementations of the current subject matter include a self-sterilization method or process to sterilize a device or container that contains an iodine-containing solution, prior to when these solutions may be used in a medical procedure to prevent Hospital-Acquired Infections (HAI) during their use. In some specific implementations, one or more of a pressure and/or temperature, and/or other parameter, are used to increase an antimicrobial activity of free iodine as an antimicrobial agent in the iodine solutions to sterilize the device or container.

In some implementations, potential reactions of molecular iodine (I₂) in water are used. Principal iodine species found in an aqueous solution include I₂, HOI, OI⁻, H₂OI⁻, I₃⁻, I⁻ and IO₃⁻:

I₃⁻→I⁻+ [symbol] (1)

HOI+H⁺+I⁻→H2O+ [symbol] (2)

Free iodine can sublime to vapor directly from the liquid iodine under stress conditions. These stress conditions can be consistent with Le Chatelier's principle, which posits that if a stress is applied to a reaction mixture at equilibrium, the net reaction goes in the direction that relieves the stress. Change in the concentration of a reactant or product is one way to place a stress on a reaction at equilibrium. Increases in temperature and pressure are other ways. Therefore, the free iodine will slowly permeate through the liquid and evaporate to the surface of the iodine surface until all of the chemical reaches equilibrium.

In implementations of the present subject matter, when device container containing the iodine solution is heated to a desired temperature, free iodine molecules will slowly increase in concentration, and when the concentration of the free iodine is above the iodine solubility in water, the free iodine will escape out of the water. By increasing the concentration of the free iodine in water and on the surface of the iodine solution, there will be more free iodine available to kill all the bacteria that may exist in the solution and in or proximate to the device. The process can include steps to heat the device in a moisture heat or dry heat medium to create more free iodine in the device container. This iodine will kill all the bacteria in the device container and will allow the device to reach a sterilization assurance level of 10⁻⁶.

In some preferred exemplary implementations, a method for self-sterilizing an iodine-containing solution includes volatilization of free iodine, which in turn includes several steps. A first step (1) includes the oxidation reaction of I₃⁻ to I₂ and I⁻ per reaction; the second step (2) the oxidation reaction of I⁻ to I₂ per reaction; the third step (3) is the evaporation of dissolved I₂ to gaseous I₂. When predicting the volatilization of iodine under various environmental conditions, it is advantageous to separately evaluate the effects of environmental factors on each step. For example, temperature may be one of the main factors that affects these three steps. With increasing temperature, the rate of I₃⁻ and I⁻ oxidation decreases, while the rate of the I2 evaporation increases.

The effects of temperature, concentration of I₂, and presence of I— on the evaporation of I₂ in I₂ and I— mixed solutions were investigated in an open and well-ventilated space. From the experiments, it was confirmed that the evaporation rate of I₂ increased exponentially as the solution temperature increased, showing a similar trend with temperature dependence of the saturated vapor pressure of I₂. The evaporation rate constant of I₂ under the temperatures of 26° C., and 80° C. in an open system is shown in the Tables below.

As temperature is increased, the concentration of I₂ in the solution also decreased very rapidly and the concentration of I₂ in vapor increased. In cases where the initial I₂ concentration was 0.99 mm, the lapsed times required to reduce less than 10% of the initial concentration were observed to be approximately 700 minutes (11.67 Hours) at 26° C., 30 minutes at 50° C., and 7 minutes at 80° C. The results indicate that the evaporation rate of I₂ is greatly influenced by the temperature. The phenomenon that I₂ evaporation was expedited at higher temperature can be explained by temperature dependency of the evaporation enthalpy and saturated vapor pressure. For an open system, the vapor pressure of elemental iodine increases about 14 torr, or 0.27 psi.

But in a closed system at constant temperature, the free energy of I₂ dissolved in water is the same as the free energy of the evaporated I₂ in the gaseous phase, when the system is in equilibrium.

I₂(soluble in water)=I₂ in Gas (3)

As shown in Table 4, a Density Calculator uses the formula p=m/V, where density (p) is equal to mass (m) divided by volume (V). Accordingly, at constant density, the pressure increases as the temperature increases.

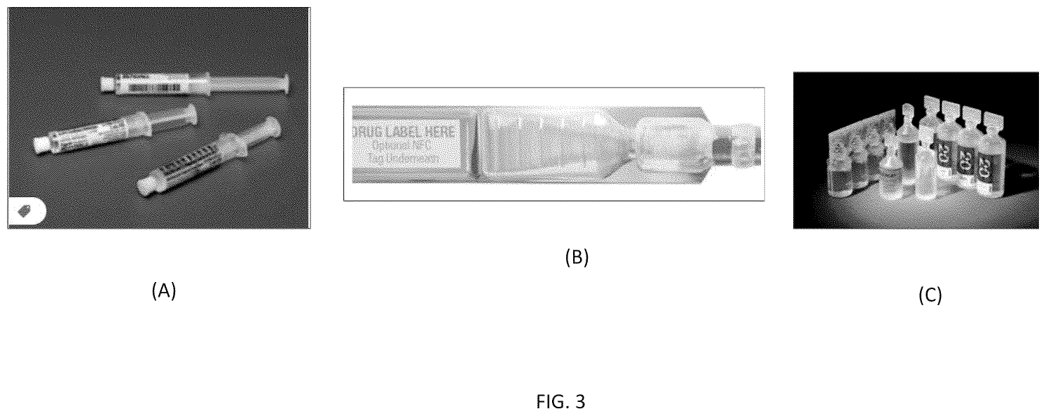

For a closed container system, such as depicted in FIGS. 3A-3C, the density does not change because the volume of the closed system is not changed, and the total mass is the same and stays in the closed system, so the density is constant. Therefore, when the temperature increases for the closed container, the pressure increases dramatically.

Accordingly, in preferred implementations, an iodine solution is contained within (i.e., inside) a closed container. At the room temperature, free elemental iodine I₂ which is soluble in the water, are the same as free elemental iodine I₂ in the gas section of the closed container. When the heating air, steam or water are introduced into the sterilization chamber process, they will slowly heat the iodine solution in the closed container. This is called the “ramp up temperature” step of the sterilization process. During this step the temperature of the iodine solution will be increase from 25° C. to 80° C., and concurrently the concentration of the free elemental iodine I₂ will increase slowly. The pressure in the closed container is increased due to more molecules of iodine evaporating from the water to gas, as well as more molecules of liquid water vapor transitioning to gas. The pressure in of the closed container system will be greater than 1 atm.

When the temperature reach to 80° C. the sterilization process will maintain the iodine solution at 80° C. for 3 to 30 minutes (depend on the prefer sterilization time setting), This step call sterilization time step. During this step, the free elemental iodine I₂, which is soluble in the water at 80° C. reach equilibrium with the free elemental iodine I₂ in the gas section of the closed container. The free iodine which is soluble in water is 290 ppm at 20° C., the free iodine will increase to 1,100 ppm at 70° C. and 1,250 ppm at 80° C.

In one example, for a closed container that contains 10 ml of iodine solution, it has approximated 2.9 ppm of free elemental iodine soluble in the container. If the iodine solution of this container is heated to 80° C., this solution will have approximate 12.5-13 ppm of free elemental iodine in the container. Because this container is a closed system and there is a fix volume therefore, the pressure of free iodine will increase dramatically proportional of the concentration of free iodine which is much larger than 13 ppm. At this high concentration of free iodine in the container, this will kill all the bacteria and virus in the container. Therefore, all the bacteria and virus will completely kill in this step.

Because all the bacteria and virus which were contaminated the iodine solution during the manufacturing process were killed by the free elemental iodine itself, so call it self-sterilization method and process. There is no other chemical was used in this sterilization process such as ETO-ethylene oxide, or hydrogen peroxide or formaldehyde etc. This sterilization process at low temperature so it will not damage the components of the device container. This self-sterilization process does not create any new impurities from the iodine solution.

After the closed container containing iodine solution was sterilized at 80° C. and the desired time (10-30 minutes), the vapor pressure of the free iodine I₂ in the container/device is very high (after the holding phase of the sterilization process). In Phase III of the sterilization process, the sterilizer is brought down to the normal room temperature and room pressure. The vapors of all elements/chemical components in the closed container will transition back to liquid form to meet the system equilibrium as expected by Le Chatelier's principle, which states that if a dynamic equilibrium is disturbed by changing the conditions, the position of equilibrium shifts to counteract the change to reestablish an equilibrium. If a chemical reaction is at equilibrium and experiences a change in pressure, temperature, or concentration of products or reactants, the equilibrium shifts in the opposite direction to offset the change.

The system of the closed container/devices must be at equilibrium at room temperature, therefore all elements/chemical components in the closed container will be transition back to liquid but due the element of iodine is solid at room temperature so all the free iodine in vapor form cannot condense to liquid form and free iodine has stay as vapor form and create a high pressure in the closed container device approximate 0.027 kpa (0.2 mmHg) I₂. Accordingly, when the container/device is unseal or opened or inject or apply the iodine vapor can escape (sublimes away) and disinfect the contact surface such as needleless valve surface, Luer lock of the catheter medical device adapter etc. and kill all the bacteria which might contaminate those surfaces. This feature is called self-decontamination.

At equilibrium, therefore, the free energy change expressed by Eq. (4) should be zero.

$$Δ {G}_{evap} ( T ) = Δ H evap ( T ) - T Δ S evap \qquad ( 4 )$$

$$= 0 ( at equilibrium in a closed system )$$

If the temperature of the system increases, the evaporation enthalpy (ΔHₑᵥₐₚ) of which the value is positive, decreases, because it is the latent heat required for the evaporation. In addition, the evaporation is a phase change reaction from liquid to gas, so the entropy change (ΔSₑᵥₐₚ) should be positive. Therefore, by increasing the temperature, both values of enthalpy (ΔHₑᵥₐₚ) and the entropy term (TΔSₑᵥₐₚ) in Eq. (4) decrease. Thus, the value of free energy change (ΔGₑᵥₐₚ) will be negative.

In summary, as the temperature increases, the equilibrium equation (4) is shifted to the right to produce more free elemental iodine and accordingly the saturated vapor pressure of free elemental I₂ increases. Therefore, the containers containing iodine solution have more free elemental iodine available to kill all the bacteria that exist in the container and make the iodine solution in the container to become sterile. The evaporation reaction of I₂ was confirmed to follow the first order reaction kinetics depending on the concentration of I₂ dissolved in the solution. As the temperature increases (to between 26° C. and 80° C.), the evaporation rate constant of free elemental iodine I₂ increases rapidly.

In some implementations, a method for sterilizing an iodine antiseptic solution includes the steps of providing the iodine antiseptic solution to a closed container, and during a sterilization cycle, heating the iodine antiseptic solution in the closed container to a sterilization temperature sufficient to generate free elemental iodine in the closed container. The method further includes the step of applying a positive pressure in the closed container to pressurize the free elemental iodine to produce sterilized iodine within the closed container. In preferred implementations, the iodine antiseptic solution includes iodine crystals and/or povidone-iodine (PVP-I).

In other implementations, a system for disinfecting epithelium of a living organism includes a device having a surface configured for being placed in proximity to the epithelium of the living organism. The device can be an indwelling urinary catheter, such as a Foley catheter or the like. The system further includes a container in contact with the device, the container containing sterilized iodine and being pressurized to a positive pressure. The container is configured to release the sterilized iodine under the positive pressure to disinfect the surface of the device prior to or while the surface is placed in proximity to the epithelium of the living organism to disinfect the epithelium in proximity thereof.

In yet other implementations, a sterilization system for sterilizing an iodine antiseptic solution includes a number of containers. Each of the plurality of closed containers contains a portion of the iodine antiseptic solution. The system further includes a chamber configured to host or mount the containers. The chamber further is configured to execute or undergo a sterilization cycle to sterilize the portion of the iodine antiseptic solution in each of the plurality of closed containers. As described herein, the sterilization cycle comprises heating the iodine antiseptic solution in each of the plurality of containers to a sterilization temperature sufficient to generate free elemental iodine in each container, and applying a positive pressure in each of the plurality of containers to pressurize the free elemental iodine to produce sterilized iodine within each container.

The chamber can be any sized vessel or stability room, in which the internal temperature can be controlled, i.e., by introduction of hot air, hot liquid, steam, or any combination thereof. For instance, the chamber can provide hot air to the containers and include a mechanism to circulate the air to ensure the hot air, liquid and/or steam is continuously in contact with each of the containers uniformly. Alternatively, the chamber can include a mechanism to provide hot air, hot liquid and/or steam directly to the portion of iodine antiseptic solution in each container, to heat the solution from room temperature to a desired sterilization temperature.

One or more containers can be closed, i.e., sealed. The containers can be formed of plastic, glass, metal or the like. The iodine antiseptic solution, in the form of an iodophor, can be placed or provided in each container, such as in a glass ampule, or form-filled and sealed in a plastic container or closed pouch.

The containers can be hosted and placed in a tray, bucket, rack or provided through the chamber in a conveyer belt or similar moving mechanism for continuous heating and/or pressurization. Automation of heating and pressurizing of the containers within the chamber allows for scaling the number of containers in which sterilized iodine is provided. In some alternative implementations, heating and pressurizing the containers can occur in two or more separate chambers or positions on an automated sterilization line.

Although a few embodiments have been described in detail above, other modifications are possible. Other embodiments may be within the scope of the following claims.

1. A method for sterilizing an iodine antiseptic solution, the iodine antiseptic solution comprising iodine crystals and/or povidone-iodine (PVP-I), the method comprising:
providing the iodine antiseptic solution to a closed container; and
during a sterilization cycle:
heating the iodine antiseptic solution in the closed container to a sterilization temperature sufficient to generate free elemental iodine in the closed container; and
applying a positive pressure in the closed container to pressurize the free elemental iodine to produce sterilized iodine within the closed container.

2. The method in accordance with claim 1, wherein the sterilization temperature is between about 60° C. to about 100° C.

3. The method in accordance with claim 2, wherein the sterilization temperature is between about 70° C. to about 80° C.

4. The method in accordance with claim 1, wherein the positive pressure applied in the closed container is greater than 1013 millibars.

5. The method in accordance with claim 1, wherein the sterilization cycle is between about 3 minutes to about 24 hours.

6. The method in accordance with claim 5, wherein the sterilization cycle is between about 10 minutes and about 30 minutes.

7. The method in accordance with claim 1, further comprising maintaining the closed container with the sterilized iodine at or near the positive pressure applied to the closed container.

8. The method in accordance with claim 1, wherein heating the iodine antiseptic solution further includes exposing the closed container to one or more of hot air, hot liquid and steam.

9. The method in accordance with claim 1, wherein heating the iodine antiseptic solution further includes exposing the iodine antiseptic solution in the closed container to one or more of hot air, hot liquid and steam.

10. The method in accordance with claim 1, further comprising sterilizing an iodine antiseptic solution in a plurality of closed containers simultaneously.

11. A system for disinfecting epithelium of a living organism, the system comprising:
a device having a surface configured for being placed in proximity to the epithelium of the living organism; and
a container in contact with the device, the container containing sterilized iodine and being pressurized to a positive pressure, the container being configured to release the sterilized iodine under the positive pressure to disinfect the surface of the device prior to or while the surface is placed in proximity to the epithelium of the living organism to disinfect the epithelium in proximity thereof.

12. The system in accordance with claim 11, wherein sterilized iodine is produced by:
providing an iodine antiseptic solution to the container; and
during a sterilization cycle:
heating the iodine antiseptic solution in the container to a sterilization temperature sufficient to generate free elemental iodine in the container; and
applying a positive pressure in the container to pressurize the free elemental iodine to produce the sterilized iodine within the container.

13. The system in accordance with claim 12, wherein the sterilization temperature is between about 60° C. to about 100° C.

14. The system in accordance with claim 13, wherein the sterilization temperature is between about 70° C. to about 80° C.

15. The system in accordance with claim 12, wherein the positive pressure applied in the closed container is greater than 1013 millibars.

16. The system in accordance with claim 12, wherein the sterilization time cycle is between about 3 minutes to about 24 hours.

17. The system in accordance with claim 16, wherein the sterilization time cycle is between about 10 minutes and about 30 minutes.

18. A sterilization system for sterilizing an iodine antiseptic solution, the iodine antiseptic solution comprising iodine crystals and/or povidone-iodine (PVP-I), the system comprising:
a plurality of containers, each of the plurality of closed containers containing a portion of the iodine antiseptic solution; and
a chamber configured to host the plurality of containers, the chamber further configured to execute a sterilization cycle to sterilize the portion of the iodine antiseptic solution in each of the plurality of closed containers, the sterilization cycle comprising:
heating the iodine antiseptic solution in each of the plurality of containers to a sterilization temperature sufficient to generate free elemental iodine in each container; and
applying a positive pressure in each of the plurality of containers to pressurize the free elemental iodine to produce sterilized iodine within each container.

19. The sterilization system in accordance with claim 18, wherein the chamber further includes a heating source for heating the iodine antiseptic solution in each of the plurality of containers, the heating source comprising one or more of hot air, hot liquid and hot steam.

20. The sterilization system in accordance with claim 18, wherein the chamber further includes a pressurization source configured to apply the positive pressure in each of the plurality of containers.

21. The sterilization system in accordance with claim 18, wherein each of the plurality of containers is a closed container, and wherein the chamber further includes a receptacle configured to host the plurality of containers, the receptacle positioning the plurality of containers for the sterilization cycle.
